(19) United States
(12) Patent Application Publication
(10) Pub. No.: US 2018/0273503 A1
(43) Pub. Date: Sep. 27, 2018

(54) NOVEL ANGIOTENSIN CONVERTING ENZYME INHIBITOR

(71) Applicant: RIKEN, Wako-shi, Saitama (JP)

(72) Inventors: Ryo NAKABAYASHI, Saitama (JP); Kazuki SAITO, Saitama (JP); Zhigang YANG, Saitama (JP); Tomoko NISHIZAWA, Saitama (JP); Tetsuya MORI, Saitama (JP)

(73) Assignee: RIKEN, Wako-shi, Saitama (JP)

(21) Appl. No.: 15/541,695

(22) PCT Filed: Jan. 6, 2016
(86) PCT No.: PCT/JP2016/050223

§ 371 Date: Jul. 5, 2017

(30) Foreign Application Priority Data
Jan. 6, 2015 (JP) 2015-000916

(51) Int. Cl. C07D 339/04; A23L 33/105; A61K 36/88; A61K 31/385

(52) U.S. Cl. CPC C07D 339/04; A23L 33/105; A61K 2236/333; A61K 31/385; A23V 2002/00; A61K 36/88

(57) ABSTRACT

This invention provides a novel compound having an activity for inhibiting an angiotensin converting enzyme (ACE). This invention relates to a compound represented by Formula (I-1), a salt thereof, or a solvate of the compound or the salt.

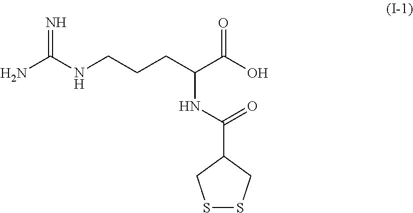 [SMILES: N=C(N)NCCCC(NC(=O)C1CSSC1)C(=O)O]

### TECHNICAL FIELD

The present invention relates to a novel compound having an activity for inhibiting an angiotensin converting enzyme. The present invention also relates to a method for producing such novel compound and applications thereof.

### BACKGROUND ART

Angiotensin is a peptide hormone having very strong activity of blood pressure elevation. There are 3 types of angiotensins; i.e., angiotensin I, angiotensin II, and angiotensin III. Angiotensin II is a peptide, which is converted from angiotensin I via cleavage of C terminal His-Leu thereof, and angiotensin III is a peptide, which is converted from angiotensin II via cleavage of N terminal Asp thereof. Among angiotensins I to III, angiotensin II is an active form, and angiotensin I is an inactive precursor thereof. Angiotensin I is primarily produced from angiotensinogen by the action of a protein degrading enzyme produced in juxtaglomerular cells of the kidney.

An enzyme that cleaves C-terminal His-Leu of angiotensin I, which is an inactive precursor, so as to convert the same into active angiotensin II in vivo is an angiotensin converting enzyme (ACE). ACE is known to mainly exist in, for example, the lung, the kidney, and the blood vessel wall of a mammalian animal, and ACE is considered to be associated with blood pressure regulation in such organs. From the viewpoint of the activity of angiotensin for blood pressure elevation, ACE has drawn attention as a target molecule of a preventive or therapeutic agent for diseases or symptoms associated with the renin-angiotensin system, such as hypertension and cardiac failure, in the field of medicine.

For example, Patent Document 1 describes a hypotensive agent comprising, as an active ingredient, at least one ingredient selected from among phenylcarboxylic acid, 5-phenyl-γ-valerolactone, and 5-phenyl-4-hydroxyvaleric acid. Patent Document 1 describes that the compound described above is capable of exerting hypotensive effects by the ACE inhibitory activity.

In the food industries, food components having ACE inhibitory activity have been searched for the purpose of the development of functional foods capable of inhibiting blood pressure elevation. Nicotianamine existing in various plants is known to have ACE inhibitory activity. Non-Patent Document 1 describes the results of investigation concerning the correlation between the nicotianamine content in vegetable food and the ACE inhibitory activity of such vegetable food.

Non-Patent Document 2 describes 2″-hydroxynicotianamine as a compound having ACE inhibitory activity contained in asparagus (Asparagus officinalis L.).

### PRIOR ART DOCUMENTS

### Patent Documents

Patent Document 1: JP 2012-144532 A

### Non-Patent Documents

Non-Patent Document 1: Hanako Izawa and Yasuo Aoyagi, Journal of the Japanese Society for Food Science and Technology, 2012, Vol. 59, No. 7, pp. 348-353
Non-Patent Document 2: Matsuda, S. and Aoyagi, Y., J. Agric. Food Chem., 2013, Vol. 61, pp. 5520-5525

### SUMMARY OF THE INVENTION

### Objects to be Attained by the Invention

As described in Non-Patent Document 1, some types of vegetable foods have ACE inhibitory activity. A majority thereof contains nicotianamine, which is a known compound having ACE inhibitory activity. Accordingly, an active substance of such vegetable food that has ACE inhibitory activity is considered to be nicotianamine. However, some vegetable foods having ACE inhibitory activity are free from or substantially free from nicotianamine. Accordingly, such vegetable foods may contain a novel compound having ACE inhibitory activity.

Under the above circumstances, it is an object of the present invention to provide a novel compound having ACE inhibitory activity.

### Means for Attaining the Objects

The present inventors have conducted concentrated studies in order to attain the above objects. As a result, the present inventors have discovered a novel compound having ACE inhibitory activity in asparagus. The present inventors have completed the present invention on the basis of the finding described above.

Specifically, the present invention is summarized as follows.

(1) A compound represented by Formula (I-1):

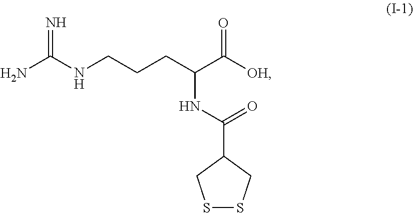 [SMILES: N=C(N)NCCCC(NC(=O)C1CSSC1)C(=O)O]

a salt thereof, or a solvate of the compound or the salt.
(2) An angiotensin converting enzyme inhibitor comprising, as an active ingredient, the compound according to (1), a salt thereof, or a solvate of the compound or the salt.
(3) A hypotensive agent comprising, as an active ingredient, the compound according to (1), a salt thereof, or a solvate of the compound or the salt.
(4) A hypotensive composition comprising, as an active ingredient, the compound according to (1), a salt thereof, or a solvate of the compound or the salt.
(5) A hypotensive food composition comprising the compound according to (1), a salt thereof, or a solvate of the compound or the salt, and one or more food industrially acceptable components.
(6) A food composition comprising the compound according to (1), a salt thereof, or a solvate of the compound or the salt in an amount of 0.2% by mass or more, relative to the total mass, and one or more food industrially acceptable components.
(7) A pharmaceutical composition comprising the compound according to (1), a salt thereof, or a solvate of the compound or the salt and one or more pharmaceutically acceptable components.
(8) A method for producing the compound according to (1) comprising:

a step of extraction comprising subjecting asparagus to extraction with a water-miscible organic solvent; and

a step of isolation comprising separating the asparagus extract obtained with a water-miscible organic solvent in the step of extraction to isolate the compound according to (1) from the extract.

(9) An asparagus extract obtained with a water-miscible organic solvent, which comprises the compound according to (1) in an amount of 0.01% to 90% by mass relative to the total mass of the extract obtained with a water-miscible organic solvent.
(10) A method for producing the asparagus extract obtained with a water-miscible organic solvent according to (9) comprising:

a step of extraction comprising subjecting asparagus to extraction with a water-miscible organic solvent.

(11) A medicament comprising, as an active ingredient, the compound according to (1), a pharmaceutically acceptable salt thereof, or a pharmaceutically acceptable solvate of the compound or the salt.
(12) The pharmaceutical composition according to (7) for use in prevention or treatment of one or more diseases or symptoms selected from the group consisting of hypertension and cardiac failure.
(13) The medicament according to (11) for use in prevention or treatment of one or more diseases or symptoms selected from the group consisting of hypertension and cardiac failure.
(14) A method for prevention or treatment of the one or more diseases or symptoms comprising administering an effective amount of the compound according to (1), a pharmaceutically acceptable salt thereof, or a pharmaceutically acceptable solvate of the compound or the salt to a subject who is in need of prevention or treatment of the diseases or symptoms.
(15) The method according to (14), wherein the diseases or symptoms are one or more diseases or symptoms selected from the group consisting of hypertension and cardiac failure.
(16) The compound according to (1), a pharmaceutically acceptable salt thereof, or a pharmaceutically acceptable solvate of the compound or the salt for use in prevention or treatment of one or more diseases or symptoms.
(17) The compound according to (16), a pharmaceutically acceptable salt thereof, or a pharmaceutically acceptable solvate of the compound or the salt, wherein the diseases or symptoms are one or more diseases or symptoms selected from the group consisting of hypertension and cardiac failure.
(18) Use of the compound according to (1), a pharmaceutically acceptable salt thereof, or a pharmaceutically acceptable solvate of the compound or the salt for the manufacture of a medicament for prevention or treatment of one or more diseases or symptoms selected from the group consisting of hypertension and cardiac failure.

### Effects of the Invention

The present invention can provide a novel compound having ACE inhibitory activity.

The objectives, the constitutions, and the effects other than those described above will be clarified in the embodiments described below.

This description includes part or all of the content as disclosed in the description and/or drawings of Japanese Patent Application No. 2015-000916, which is a priority document of the present application.

### BRIEF DESCRIPTION OF THE DRAWINGS

FIG. 1 shows a summary of the results of purification and isolation of a novel compound; i.e., asparaptine, from an asparagus spear. A: a scheme of the purification; and B: an MS chromatogram of preparative ODS-LC-MS with the use of the preparative LC-PDA-MS system, which is the final process of the purification.

FIG. 2 shows the one-dimensional NMR spectra of asparaptine. A: ¹H-NMR spectra; B: ¹³C-NMR spectra.

FIG. 3 shows the results of HPLC analysis to determine the absolute configuration of asparaptine at the alpha carbon position of arginine.

### EMBODIMENTS FOR CARRYING OUT THE INVENTION

Hereafter, preferable embodiments of the present invention are described in greater detail.

### [1. Novel Compound Having ACE Inhibitory Activity]

In the present invention, the term “angiotensin converting enzyme (ACE)” refers to an enzyme that converts angiotensin, which is a peptide hormone having very strong activity of blood pressure elevation, from a form of inactive angiotensin I into a form of active angiotensin II. ACE cleaves C terminal His-Leu of angiotensin I so as to catalyze a reaction of angiotensin II production. ACE is considered to be involved in blood pressure regulation in organs, such as the lung, the kidney, and the blood vessel walls of mammalian animals, through angiotensin conversion. In the present invention, “ACE inhibitory activity” is an activity that substantially inhibits the progress of the enzymatic reaction of ACE as described above. For example, such activity substantially inhibits the progress of a reaction of cleaving C terminal His-Leu of angiotensin I that is catalyzed by ACE. A compound having ACE inhibitory activity is capable of substantially inhibiting the production of active angiotensin II.

In the present invention, “ACE inhibitory activity” inhibits, for example, the progress of an enzymatic reaction of ACE by 10% or more, such as 20% or more, preferably 40% or more, more preferably 50% or more, further preferably 80% or more, still further preferably 90% or more, and particularly preferably 95% or more. ACE inhibitory activity can be quantitatively evaluated with the use of, for example, a commercially available kit for ACE inhibitory activity assay. Alternatively, quantitative evaluation can be performed with reference to known literature, such as Cheung, H. S. and Cuchman, D. W., Biochim. Biophys. Acta, 1973, Vol. 293, pp. 451-463; Hanako Izawa and Yasuo Aoyagi, Journal of the Japanese Society for Food Science and Technology, 2012, Vol. 59, No. 7, pp. 348-353; or Matsuda, S. and Aoyagi, Y., J. Agric. Food Chem., 2013, Vol. 61, pp. 5520-5525. For example, ACE inhibitory activity of a target sample (e.g., a compound) can be evaluated with the use of a commercially available kit for ACE inhibitory activity assay (ACE Kit-WST, A502, Dojindo Laboratories) in the manner described below. In principle, such kit enzymatically detects 3-hydroxybutyric acid (3HB) cleaved from 3-hydroxybutyryl-glycyl-glycyl-glycine (3HB-GGG) by ACE activity. In accordance with the instructions of the kit, a sample solution containing a test compound at a given concentration is prepared in a reaction vessel, and a control solution is prepared with the addition of the same volume of water instead of the sample solution in another reaction vessel. An enzyme reaction solution is added to each reaction vessel, and the reaction is then allowed to proceed, respectively. As a blank sample, a solution prepared with the addition of the same volume of water instead of the enzyme solution is subjected to the same treatment. The absorbance (450 nm) of the chromogenic indicator used in the enzymatic method generated together with 3HB cleaved from 3HB-GGG by ACE activity is measured. On the basis of the values measured for the sample, the control sample, and the blank sample, ACE inhibitory activity of each sample is determined using the formula shown below. Alternatively, ACE inhibitory activity of the target sample can be evaluated in the manner described below. A substrate peptide that is a mimic of a C-terminal region of angiotensin I (e.g., hippuryl-histidyl-leucine (HHL)) and a sample are mixed with each other in an appropriate buffer solution. ACE is added to the resulting mixture and an enzymatic reaction is performed under appropriate conditions (e.g., 37° C. and pH 8.3) for a given period of time. After the enzymatic reaction is terminated, a product generated from the substrate peptide (e.g., hippuric acid in the case of HHL) is quantitatively measured via analytical means, such as absorbance measurement, immunoanalysis such as ELISA or RIA, or chromatography analysis such as HPLC or LC-MS. A sample-free control and an ACE-free blank are subjected to the same experiment. On the basis of the values measured for the sample, the control sample, and the blank sample, ACE inhibitory activity of each sample is determined using the formula shown below. Also, a dose-response curve for ACE activity of each sample may be prepared to determine the half maximal inhibitory concentration (IC₅₀ value) of each sample.

ACE inhibitory activity (%)={(blank value−sample value)/(control value−blank value)}×100

The present inventors focused on the fact that an extract obtained from an asparagus spear has ACE inhibitory activity. The present inventors had made concentrated studies and, as a consequence, the present inventors have discovered the presence of a novel compound having ACE inhibitory activity in asparagus.

The present invention relates to a compound represented by Formula (I-1):

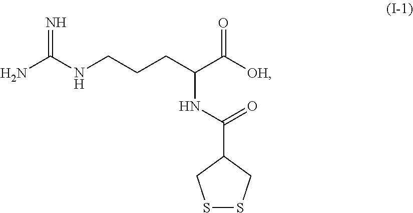 [SMILES: N=C(N)NCCCC(NC(=O)C1CSSC1)C(=O)O]

a salt thereof, or a solvate of the compound or the salt. A compound represented by Formula (I-1) is a novel compound having a structure resulting from an amide bond formed between an α-amino group of arginine and a carboxyl group of asparagusic acid (Dawid, C. and Hofmann, T., J. Agric. Food Chem., 2012, Vol. 60, pp. 11877-11888), which are known compounds.

A compound represented by Formula (I-1) has two stereocenters (chiral centers). In the present invention, a compound represented by Formula (I-1) encompasses enantiomers and diastereomers of such compound and a mixture thereof such as a racemate. In the present invention, a compound represented by Formula (I-1) is preferably a compound represented by Formula (I-1S):

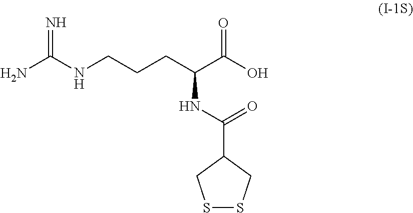 [SMILES: N=C(N)NCCC[C@H](NC(=O)C1CSSC1)C(=O)O]

having an absolute configuration. In this description, a compound represented by Formula (I-1) or (I-1S) is occasionally expressed as “asparaptine.” A compound represented by Formula (I-1) or (I-1S) has ACE inhibitory activity. Accordingly, a compound represented by Formula (I-1) or (I-1S) can be used as an active ingredient for inhibiting ACE in vivo or in vitro.

In the present invention, a compound represented by Formula (I-1) or (I-1S) also encompasses a salt thereof. Preferable examples of salts of the compound represented by Formula (I-1) or (I-1S) according to the present invention include, but are not limited to: cations, such as sodium ion, potassium ion, calcium ion, magnesium ion, and substituted or unsubstituted ammonium ion; and anions, such as chloride ion, bromide ion, formate ion, acetate ion, maleate ion, fumarate ion, benzoate ion, ascorbate ion, pamoate ion, succinate ion, bismethylene salicylate ion, methanesulfonate ion, ethanedisulfonate ion, propionate ion, tartrate ion, salicylate ion, citrate ion, gluconate ion, aspartate ion, stearate ion, palmitate ion, itaconate ion, glycolate ion, p-aminobenzoate ion, glutamate ion, benzenesulfonate ion, cyclohexyl sulfamate ion, methanesulfonate ion, ethanesulfonate ion, isethionate ion, benzenesulfonate ion, p-toluenesulfonate ion, naphthalenesulfonate ion, phosphate ion, nitrate ion, sulfate ion, carbonate ion, bicarbonate ion, and perchlorate ion. When a compound represented by Formula (I-1) or (I-1S) of the present invention is in the form of the salt, such compound can be used without substantially deteriorating ACE inhibitory activity.

In the present invention, a compound represented by Formula (I-1) or (I-1S) also encompasses a solvate of the compound or the salt thereof. Preferable examples of a solvent that can form a solvate with the compound or the salt thereof include, but are not limited to, an organic solvent, such as a lower alcohol (e.g., an alcohol having 1 to 6 carbon atoms, such as methanol, ethanol, propanol (e.g., 2-propanol), and butanol (e.g., n-butanol)), a higher alcohol (e.g. an alcohol having 7 or more carbon atoms, such as 1-heptanol and 1-octanol), aliphatic ketone (e.g., acetone), aliphatic sulfoxide (e.g., dimethyl sulfoxide (DMSO)), and ester (e.g., ethyl acetate); and water; and a mixture of any thereof. When the compound represented by Formula (I-1) or (I-1S) of the present invention or a salt thereof is in the form of a solvate with the solvent, such compound can be used without substantially deteriorating ACE inhibitory activity.

In the present invention, a compound represented by Formula (I-1) or (I-1S) also encompasses a protected form thereof. The term “protected form” used herein refers to a form resulting from introduction of a protecting group into one or more functional groups (e.g., a hydroxyl or carboxylic acid group). The term “protecting group” used herein refers to a group that is to be introduced into a particular functional group, so as to prevent an undesirable reaction from proceeding. Such group is quantitatively removed under particular reaction conditions, and it is substantially stable: i.e., unreactive, under other reaction conditions. Examples of protecting groups capable of forming protective forms of the compounds are not particularly limited. Preferably, examples of carboxylic acid protecting groups include alkyl ester (e.g., methyl, ethyl, or isopropyl ester) and aryl alkyl ester (e.g., benzyl ester), examples of amino protecting groups include alkoxy amide (t-butoxycarbonyl (Boc)) and aryl alkyl amide (e.g., fluorenylmethyloxycarbonyl (Fmoc) or benzyloxycarbonyl (Z)), and an example of a guanidino protecting group is sulfonyl amide (e.g., p-toluenesulfonyl (Tos)), although examples are not limited thereto. Protection with the protecting groups and deprotection of such protecting groups can be performed under conventional reaction conditions. When the compound represented by Formula (I-1) or (I-1S) of the present invention is protected with the protecting group, such protected form of the compound can be used without substantially deteriorating ACE inhibitory activity.

In the present invention, a compound represented by Formula (I-1) or (I-1S) also encompasses a prodrug form thereof. The term “prodrug” used herein refers to a compound that is converted into a parent drug in vivo. Prodrug forms of the compounds are not limited, and examples thereof include an ester made from a carboxylic acid and an alcohol and sulfonyl amide made from a guanidino group and a sulfonic acid. When the compound represented by Formula (I-1) or (I-1S) of the present invention is in the form of the prodrug described above, pharmacokinetics of a prodrug at the time of administration thereof to the target can be enhanced without substantially deteriorating ACE inhibitory activity of a parent drug; i.e., a compound represented by Formula (I-1) or (I-1S).

Since the compound represented by Formula (I-1) or (I-1S) of the present invention has the properties as described above, it can exert ACE inhibitory activity.

### [2. Method for Producing Novel Compound]

The present invention also relates to a method for producing the compound represented by Formula (I-1) or (I-1S) of the present invention.

The compound represented by Formula (I-1) or (I-1S) of the present invention has a structure resulting from an amide bond formed between an α-amino group of arginine and a carboxyl group of asparagusic acid. Also, the compound represented by Formula (I-1) or (I-1S) of the present invention is present as a naturally occurring substance in asparagus. Accordingly, the compound represented by Formula (I-1) or (I-1S) of the present invention may be produced by means of purification and isolation of a naturally occurring substance contained in asparagus, or it may be produced by a synthetic means from arginine, asparagusic acid, or a precursor compound thereof. Both embodiments are within the scope of the method of production according to the present invention. Hereafter, these embodiments are described in detail.

### [2-1. Method of Production by Means of Purification and Isolation of Naturally Occurring Substance]

When the method for producing the compound represented by Formula (I-1) or (I-1S) according to an embodiment of the present invention is carried out by means of purification and isolation of a naturally occurring substance, the method of the present invention comprises: a step of extraction comprising subjecting asparagus to extraction with a water-miscible organic solvent; and a step of isolation comprising separating an asparagus extract obtained with a water-miscible organic solvent in the step of extraction to isolate the compound represented by Formula (I-1) or (I-1S) of the present invention from the extract.

Hereafter, these steps are described.

Asparagus used in the step of extraction may be a plant of asparagus existing in nature, or that is cultivated for food, pharmaceutical, or horticultural applications. Specific intraspecific classifications (e.g., subspecies or varieties), raising or cultivation areas, and cultivation conditions (e.g., light/dark conditions) thereof are not particularly limited. Examples of asparagus plants used in this step include various plants of Asparagus, such as A. officinalis as a cultivated crop, A. schoberioides as a wild species, and A. cochinchinensis, A. plumosus, A. asparagoides, and A. myriocladus as foliage plants. In the present invention, accordingly, the term “Asparagus” encompasses a wide variety of plants of Asparagus including those exemplified above, as well as A. officinalis as a cultivated crop. Use of white asparagus grown in the dark or green asparagus grown in the light is preferable because of a large content of the compound represented by Formula (I-1) or (I-1S) of the present invention. Also, use of above ground parts (e.g., spears, stems, pseudo leaves, leaves, fruits, or seeds) or underground parts (e.g., rhizomes) of the asparagus is preferable because of a large content of the compound represented by Formula (I-1) or (I-1S) of the present invention, and the use of an asparagus spear with the full-length of the above ground part of 5 to 15 cm is more preferable. When an asparagus spear having the features described above is used, a region of 1 to 5 cm from the apex is preferably used, and a region of 1 to 3 cm from the apex is more preferably used. With the use of asparagus that satisfies the conditions described above, the compound represented by Formula (I-1) or (I-1S) of the present invention can be produced with a high yield.

In the step of extraction, a fresh asparagus plant that satisfies the conditions described above or a part of a fresh plant obtained by cutting the same may be used in that state (i.e., in a fresh state), or the fresh plant or a part thereof may be dehydrated by means of, for example, drying via heating or lyophilizing and used in a dried state. The asparagus in either the fresh or dry state may be subjected to extraction in that state, or small pieces or powders obtained via cutting or grinding may be subjected to extraction. Both means are within the scope of the embodiments of the step of extraction.

Examples of water-miscible organic solvents used in the step of extraction include lower aliphatic alcohol, aliphatic ketone, aliphatic sulfoxide, a halogen-containing organic solvent, and a mixture of any thereof. The water-miscible organic solvent is preferably methanol, ethanol, propanol (e.g., 2-propanol), butanol (e.g., n-butanol), acetone, dimethyl sulfoxide (DMSO), or a mixture of any thereof. The water-miscible organic solvent may occasionally be in the form of a solvent mixture with water. Examples of particularly preferable water-miscible organic solvents include a hydrous lower aliphatic alcohol (an aqueous solution comprising preferably 70% to 90% and more preferably 80% lower aliphatic alcohol (e.g., methanol or ethanol) by mass). The water-miscible organic solvent is used in an amount of 10 ml or more, preferably 40 ml or more, and more preferably 50 ml or more to 1,000 ml or less, and preferably 100 ml or less, relative to 1 g of asparagus to be extracted. The asparagus extract obtained with the use of the water-miscible organic solvent comprises the compound represented by Formula (I-1) or (I-1S) of the present invention in a concentrated state in an amount of generally 0.01% by mass or more, for example, 0.01% to 90% by mass and typically 0.05% to 90% by mass, relative to the total mass of the extract obtained with the water-miscible organic solvent. In a particular embodiment, the asparagus extract obtained with a water-miscible organic solvent comprises the compound represented by Formula (I-1) or (I-1S) of the present invention in a highly concentrated state in an amount of generally 0.2% by mass or more, for example, 0.2% to 90% by mass, typically 0.5% to 90% by mass, and particularly 1% to 90% by mass, relative to the total mass of the extract obtained with the water-miscible organic solvent. With the use of the water-miscible organic solvent, accordingly, the compound represented by Formula (I-1) or (I-1S) of the present invention can be efficiently extracted into a fraction of the water-miscible organic solvent.

The step of extraction is preferably carried out at room temperature to reflux temperature, more preferably 10° C. to 40° C., and further preferably 20° C. to 30° C. The duration of extraction is preferably 1 hour to several days (e.g., for 10 days), more preferably 1 hour to 1 day, and further preferably 1 hour to overnight (e.g., for 12 hours). By performing extraction under the conditions described above, the compound represented by Formula (I-1) or (I-1S) of the present invention can be efficiently extracted into a fraction of the water-miscible organic solvent.

In the step of isolation, a means for separating the asparagus extract obtained with a water-miscible organic solvent in the step of extraction is not particularly limited. Various means for separation that are generally used in the art, for example, liquid-liquid partitioning, various column chromatography techniques, such as partition, normal phase, reverse phase, ion-exchange, or gel filtration chromatography, high-performance liquid chromatography (HPLC), distillation, dialysis, ultrafiltration, or recrystallization, can be employed. It is preferable that the step of isolation further comprise: a step of lipophilic component removal comprising removing a lipophilic component partitioned into an organic phase as a result of liquid-liquid partitioning of the asparagus extract obtained with a water-miscible organic solvent; and step of preparative chromatography comprising separating the extract of the water-soluble component obtained in the step of lipophilic component removal via several steps of chromatography processes (e.g., reversed phase open column chromatography, reversed phase HPLC, or reversed phase LC-MS) to obtain a preparative chromatography fraction containing the target compound. In the step of lipophilic component removal, an organic phase used for liquid-liquid partitioning is preferably n-hexane, chloroform or ethyl acetate, or a combination of any thereof. With the use of the organic phase, a lipophilic component, such as a lipid and/or chlorophyll, can be removed. An extract of a water-soluble component obtained thereby comprises the compound represented by Formula (I-1) or (I-1S) of the present invention in a concentrated state, generally in an amount of 0.01% by mass or more, for example, 0.01% to 90% by mass, and typically 0.05% to 90% by mass, relative to the total mass of the extract of the water-soluble component. In a particular embodiment, the extract of the water-soluble component comprises the compound represented by Formula (I-1) or (I-1S) of the present invention in a highly concentrated state, generally in an amount of 0.2% by mass or more, for example, 0.2% to 90% by mass, typically 0.5% to 90% by mass, and particularly 1% to 90% by mass, relative to the total mass of the extract of the water-soluble component. When a support used for reverse phase chromatography, reverse phase HPLC, and reverse phase LC-MS in the step of preparative chromatography is ODS, a mobile phase is preferably a methanol-aqueous system or an acetonitrile-aqueous system containing acid (e.g., 0.1% HCOOH by volume). The mobile phase described above may be used under either an isocratic elution or linear gradient elution conditions. When the mobile phase described above is used, a target compound can be obtained in a fraction of an aqueous solution of 10% to 20% by volume of methanol or an aqueous solution of 90% to 100% by volume of acetonitrile (containing an acid (e.g., 0.1% HCOOH by volume)). By performing the step of isolation by the means described above, accordingly, the compound represented by Formula (I-1) or (I-1S) of the present invention can be obtained from the asparagus extract obtained with a water-miscible organic solvent in a concentrated or isolated state.

### [2-2. Method of Production Via Synthetic Means]

In an embodiment via synthetic means, the method of production of the present invention comprises a step of coupling comprising subjecting arginine or a protected form thereof to a coupling reaction with asparagusic acid to form a compound represented by Formula (I-1) or (I-1S). In the step of coupling, a means for subjecting arginine or a protected form thereof to a coupling reaction with asparagusic acid is not particularly limited. A reaction of amide bond formation that is generally used in the art in which an acid halide (e.g., acid chloride), an activated ester (e.g., N-hydroxysuccinimide ester), or the like is used as an activated carboxyl group of asparagusic acid can be used. In such a case, arginine is preferably in a protected form, such that a protecting group has been introduced into a C-terminal carboxyl group and a side-chain guanidino group.

Arginine and asparagusic acid used in the step of coupling may be obtained by means of purification and isolation of naturally occurring substances in accordance with a conventional technique, or compounds produced in advance may be purchased and used. When arginine is used in its protected form, a protecting group may be introduced into arginine in accordance with a conventional technique, so as to convert arginine into a protected form, or a protected form of arginine that has been produced in advance may be purchased and used. Both alternatives are within the scope of the step of coupling.

### [3. Application of Novel Compound Having ACE Inhibitory Activity]

The compound represented by Formula (I-1) or (I-1S) of the present invention has ACE inhibitory activity. ACE is considered to be involved in blood pressure regulation through angiotensin conversion. Accordingly, a compound having ACE inhibitory activity can be used as an active ingredient of a pharmaceutical product or a food product used for blood pressure reduction or prevention or treatment of diseases or symptoms associated with the renin-angiotensin system, such as hypertension or cardiac failure. Accordingly, an aspect of the present invention relates to an ACE inhibitor comprising, as an active ingredient, the compound represented by Formula (I-1) or (I-1S) of the present invention. Also, another aspect of the present invention relates to a hypotensive agent comprising, as an active ingredient, the compound represented by Formula (I-1) or (I-1S) of the present invention. Further, another aspect of the present invention relates to a hypotensive composition comprising, as an active ingredient, the compound represented by Formula (I-1) or (I-1S) of the present invention. A further aspect of the present invention relates to a food composition comprising the compound represented by Formula (I-1) or (I-1S) of the present invention and one or more food industrially acceptable components. Furthermore, the present invention relates to a medicament comprising, as an active ingredient, the compound represented by Formula (I-1) or (I-1S) of the present invention, or a pharmaceutical composition comprising the compound represented by Formula (I-1) or (I-1S) of the present invention and one or more pharmaceutically acceptable components. The ACE inhibitor according to the present invention can be used for ACE inhibition in vivo or in vitro. The food composition, the medicament, and the pharmaceutical composition according to the present invention can be used for blood pressure reduction or prevention or treatment of diseases or symptoms associated with the renin-angiotensin system, such as hypertension or cardiac failure, by the ACE inhibitory activity of the compound represented by Formula (I-1) or (I-1S) of the present invention contained as an active ingredient.

In the ACE inhibitor, the food composition, the medicament, and the pharmaceutical composition according to the present invention, the compound represented by Formula (I-1) or (I-1S) of the present invention contained as an active ingredient may be incorporated in a pure state (i.e., in an isolated state) or in a partially purified state. When the compound represented by Formula (I-1) or (I-1S) of the present invention is incorporated in a pure state, the compound can be used in the pure state achieved by performing the method of production according to the present invention as described above. When the compound represented by Formula (I-1) or (I-1S) of the present invention is incorporated in a partially purified state, the extract or the fraction obtained in the step of the method of production according to the present invention by means of purification and isolation of a naturally occurring substance can be used in a partially purified state of the compound. In such a case, the asparagus extract obtained with a water-miscible organic solvent or the fraction of the water-miscible organic solvent obtained in the step of extraction, an extract of a water-soluble component or an aqueous fraction obtained in the step of lipophilic component removal, or a fraction obtained in the step of preparative chromatography is preferably used in a partially purified state of the compound represented by Formula (I-1) or (I-1S) of the present invention. By incorporating the compound represented by Formula (I-1) or (I-1S) of the present invention in the manner as described above, the ACE inhibitor, the food composition, the medicament, and the pharmaceutical composition according to the present invention can exert ACE inhibitory activity.

In general, the ACE inhibitor and the food composition according to the present invention comprise the compound represented by Formula (I-1) or (I-1S) of the present invention in an amount effective for ACE inhibition. For example, an amount effective for ACE inhibition is generally 0.2% by mass or more, such as 0.2% to 90% by mass, and it is preferably 0.5% to 90% by mass, and more preferably 1% to 90% by mass, relative to the total mas of the ACE inhibitor or the food composition. When the ACE inhibitor according to the present invention comprises the compound represented by Formula (I-1) or (I-1S) of the present invention in the effective amount described above, it can exert ACE inhibitory activity in vivo or in vitro. The food composition according to the present invention can exert ACE inhibitory activity in the body of a person who had ingested the food composition.

The food composition according to the present invention can be processed into various forms of food products that are generally used in the art, and examples thereof include solids (e.g., powders, tablets, and granules), pastes, and liquids. The food composition according to the present invention may comprise, in addition to the active ingredients described above, one or more food components and one or more food industrially acceptable preservatives, stabilizers, dispersants, swelling agents, surfactants, oil liquids, buffers, antioxidants, sweetening agents, flavoring agents, dyes, and pigments.

The food composition according to the present invention may be used for a food product without any further processing, or it may be used as a raw material for food in the form of a mixture thereof with other foods or food components. The food composition according to the present invention may be in the form of a general food or beverage product, and it may also be in the form of, for example, a dietary supplement. Examples of forms of dietary supplements include powders, granules, tablets optionally provided with sugar coats or soluble coats, capsules, elixirs, microcapsules, syrups, and suspensions. Examples of additives that can be incorporated into tablets or capsules include, but are not limited to, binders, such as gelatin, corn starch, gum tragacanth, and gum arabic, excipients, such as crystalline cellulose, swelling agents, such as alginic acid, corn starch, and gelatin, lubricants, such as magnesium stearate, sweetening agents, such as sucrose, lactose, and saccharin, and flavoring agents, such as peppermint, Gaultheria adenothrix (Akamono) oil, and cherry. When a preparation is a capsule, the preparation may further contain a liquid carrier such as oil and fat.

The compound represented by Formula (I-1) or (I-1S) of the present invention as an active ingredient of the food composition according to the present invention exists as a naturally occurring substance in asparagus. Accordingly, a food composition containing the compound represented by Formula (I-1) or (I-1S) of the present invention is safe and low in toxicity. Because of such properties, the food composition according to the present invention can be used without substantially affecting health conditions of a subject who ingests the same.

The compound represented by Formula (I-1) or (I-1S) of the present invention as an active ingredient of the food composition according to the present invention can be used for blood pressure reduction by the ACE inhibitory activity. Accordingly, an embodiment of the present invention relates to a hypotensive food composition comprising the compound represented by Formula (I-1) or (I-1S) of the present invention and one or more food industrially acceptable components. Through ingestion of the hypotensive food composition according to the present invention, the blood pressure level can be lowered by the ACE inhibitory activity in the body of a subject who ingests the same.

The present invention also relates to a medicament comprising, as an active ingredient, the compound represented by Formula (I-1) or (I-1S) of the present invention, or a pharmaceutical composition comprising the compound represented by Formula (I-1) or (I-1S) of the present invention and one or more pharmaceutically acceptable components. The compound represented by Formula (I-1) or (I-1S) of the present invention as an active ingredient of the medicament or pharmaceutical composition of the present invention exists as a naturally occurring substance in asparagus. Accordingly, the medicament or pharmaceutical composition comprising the compound represented by Formula (I-1) or (I-1S) of the present invention is safe and low in toxicity. Because of such properties, the medicament or pharmaceutical composition of the present invention can be administered to a wide variety of targets. Examples of the targets include subjects and patients of humans and non-human mammalians (e.g., warm-blooded animals, such as pigs, dogs, cows, rats, mice, guinea pigs, rabbits, chickens, sheeps, cats, monkeys, hamadryas baboons, and chimpanzees). By administering the medicament or pharmaceutical composition of the present invention to the targets, blood pressure reduction or prevention or treatment of diseases or symptoms associated with the renin-angiotensin system, such as hypertension and cardiac failure, can be realized.

The term “prevention” used herein refers to substantial inhibition of the occurrence (i.e., crisis or onset) of the diseases and/or symptoms. The term “treatment” used herein refers to suppression (e.g., suppression of advancement), remission, recovery, and/or treatment of the diseases and/or symptoms that had occurred (i.e., crisis or onset of the diseases and/or symptoms).

When the medicament or pharmaceutical composition of the present invention is administered to a target (a human patient, in particular), a therapeutically effective dose and number of doses should be accurately and definitely determined by a primary doctor based on many factors, such as the age of the target, the sexuality of the target, an accurate condition (e.g., severity) of a disease and/or symptom to be prevented or treated, and a route of administration. Accordingly, the medicament or pharmaceutical composition of the present invention generally comprises a therapeutically effective amount of the compound represented by Formula (I-1) or (I-1S) of the present invention as an active ingredient. A therapeutically effective amount is generally 0.01% by mass or more, and, for example, it is 0.01% to 90% by mass, preferably from 0.05% to 90% by mass, more preferably from 0.1% to 90% by mass, further preferably from 0.2% to 90% by mass, still further preferably from 0.5% to 90% by mass, and particularly preferably 1% to 90% by mass, relative to the total mass of the medicament or pharmaceutical composition. By comprising the therapeutically effective amount of the compound represented by Formula (I-1) or (I-1S) of the present invention, the medicament or pharmaceutical composition of the present invention can exert desirable therapeutic effects in the body of a target to which the medicament or pharmaceutical composition had been administered by the ACE inhibitory activity.

The medicament or pharmaceutical composition of the present invention can be formulated into various dosage forms commonly used in the art in accordance with a route of administration of interest. In addition to the components described above, the medicament or pharmaceutical composition of the present invention may comprise one or more pharmaceutically acceptable carriers, excipients, binders, dispersants, vehicles, solubilizers, preservatives, stabilizers, swelling agents, lubricants, surfactants, oil liquids, buffers, soothing agents, antioxidants, sweetening agents, and flavoring agents.

The medicament or pharmaceutical composition of the present invention can generally be used for oral or parenteral administration. Examples of formulations used for oral administration include powders, tablets optionally provided with sugar coats or soluble coats, capsules, elixirs, microcapsules, syrups, and suspensions. Examples of additives that can be incorporated into tablets or capsules include, but are not limited to, binders, such as gelatin, corn starch, gum tragacanth, and gum arabic, excipients, such as crystalline cellulose, swelling agents, such as alginic acid, corn starch, and gelatin, lubricants, such as magnesium stearate, sweetening agents, such as sucrose, lactose, and saccharin, and flavoring agents, such as peppermint, Gaultheria adenothrix (Akamono) oil, and cherry. When a formulation is a capsule, the formulation may further contain a liquid carrier such as oil and fat. Examples of formulations used for parenteral administration include an injection formulation comprising an aseptic solution or suspension of the medicament or pharmaceutical composition of the present invention and water or other pharmaceutically acceptable liquid. Examples of additives that can be incorporated into injection formulations include, but are not limited to, vehicles such as an isotonic solution containing physiological saline, glucose and other adjuvants (e.g., D-sorbitol, D-mannitol, and sodium chloride), solubilizers such as alcohol (e.g., ethanol and benzyl alcohol), ester (e.g., benzyl benzoate) and polyalcohol (e.g., propylene glycol and polyethylene glycol), nonionic surfactants such as polysorbate 80 and polyoxyethylene hydrogenated castor oil, oil liquids such as sesame oil and soybean oil, buffers such as phosphate buffer and sodium acetate buffer, soothing agents such as benzalkonium chloride and procaine hydrochloride, stabilizers such as human serum albumin and polyethylene glycol, preservatives, and antioxidants. The prepared injection formulations are generally filled into appropriate vials (e.g., ampoules) and stored in an appropriate environment before use.

The medicament or pharmaceutical composition of the present invention can be formulated into the form of a depot formulation used for parenteral administration. In such a case, the medicament or pharmaceutical composition of the present invention in the form of a depot formulation may be embedded hypodermically or intramuscularly, or administered via intramuscular injection. By formulating the medicament or pharmaceutical composition of the present invention into the form of a depot formulation, the compound represented by Formula (I-1) or (I-1S) of the present invention as an active ingredient can be released continuously over a long period of time.

The medicament or pharmaceutical composition of the present invention may be in the form of a unit-dose formulation or a multiple-dose formulation. The route of administration and the number of administration of the medicament or pharmaceutical composition of the present invention are not particularly limited, and the medicament or pharmaceutical composition may be administered in a single application or multiple applications orally or parenterally.

The compound represented by Formula (I-1) or (I-1S) of the present invention can be used for prevention or treatment of a target who has the diseases or symptoms associated with the renin-angiotensin system described above (e.g., hypertension or cardiac failure). Accordingly, an embodiment of the present invention relates to a method for prevention or treatment of one or more diseases or symptoms associated with the renin-angiotensin system described above comprising administering an effective amount of the compound represented by Formula (I-1) or (I-1S) of the present invention, a pharmaceutically acceptable salt thereof, or a pharmaceutically acceptable solvate of the compound or the salt to a target who is in need of prevention or treatment of such diseases or symptoms. The diseases or symptoms associated with the renin-angiotensin system are preferably one or more diseases or symptoms selected from the group consisting of hypertension and cardiac failure. Administration of the compound represented by Formula (I-1) or (I-1S) of the present invention to a target who is in need of prevention or treatment of one or more diseases or symptoms associated with the renin-angiotensin system enables prevention or treatment of such diseases or symptoms by the ACE inhibitory activity of the compound represented by Formula (I-1) or (I-1S) of the present invention.

Another embodiment of the present invention relates to the compound represented by Formula (I-1) or (I-1S) of the present invention, a pharmaceutically acceptable salt thereof, or a pharmaceutically acceptable solvate of the compound or the salt for use in blood pressure reduction or prevention or treatment of one or more diseases or symptoms associated with the renin-angiotensin system described above (e.g., hypertension or cardiac failure). Another embodiment of the present invention relates to use of the compound represented by Formula (I-1) or (I-1S) of the present invention, a pharmaceutically acceptable salt thereof, or a pharmaceutically acceptable solvate of the compound or the salt for the manufacture of a medicament for blood pressure reduction or prevention or treatment of one or more diseases or symptoms associated with the renin-angiotensin system described above (e.g., hypertension or cardiac failure). The diseases or symptoms associated with the renin-angiotensin system are preferably one or more diseases or symptoms selected from the group consisting of hypertension and cardiac failure. The use of the medicament of the present invention for blood pressure reduction or prevention or treatment of one or more diseases or symptoms associated with the renin-angiotensin system enables blood pressure reduction or prevention or treatment of the diseases or symptoms by the ACE inhibitory activity of the compound represented by Formula (I-1) or (I-1S) of the present invention.

### EXAMPLES

Hereafter, the present invention is described in greater detail with reference to the examples, although the technical scope of the present invention is not limited to these examples.

### [I: Purification and Isolation of a Novel Compound]

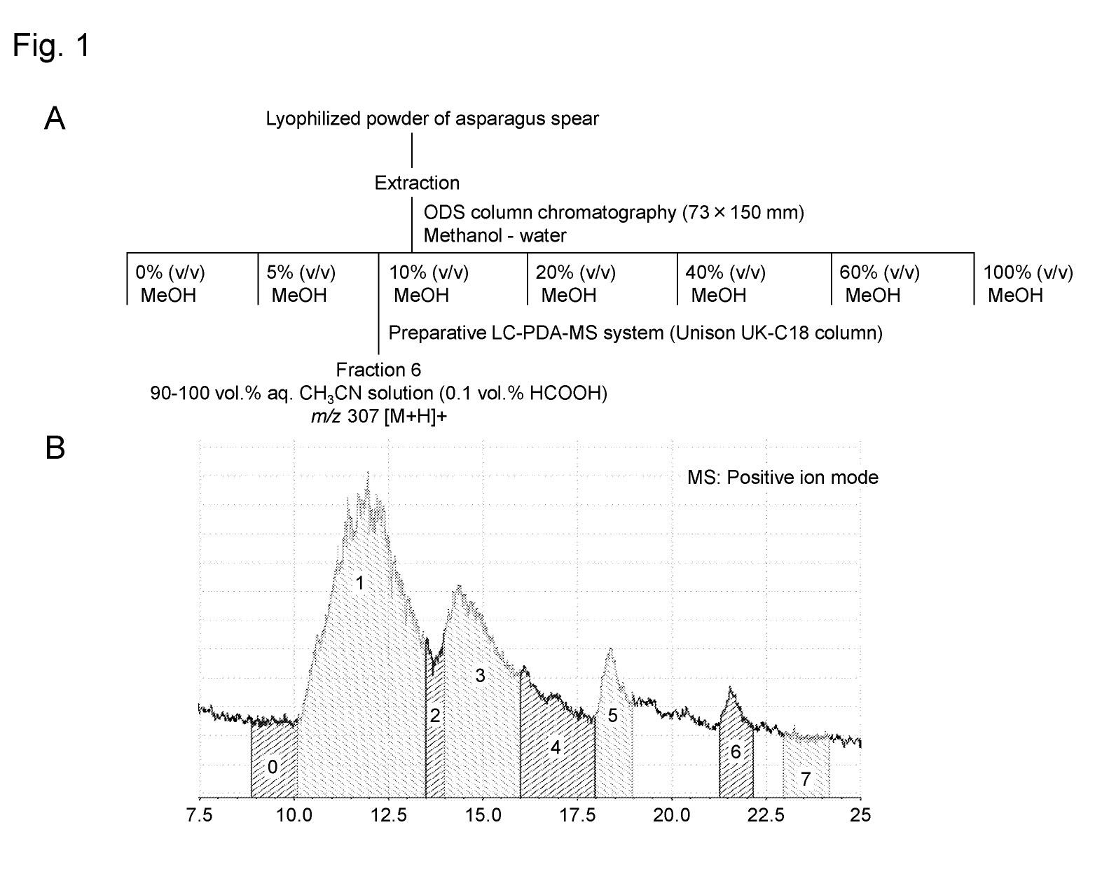

Spears (the full length of the above ground part: about 15 cm, 970.7 g) of fresh green asparagus (Asparagus officinalis L.) were purchased from a retail store, and the spears were cut into small pieces of 2 to 3 mm with the use of a ceramic knife. The small pieces were rapidly frozen in liquid nitrogen. The frozen small pieces were lyophilized and then grounded. The lyophilized asparagus powder (57.5 g) was subjected to extraction with the use of an aqueous solution of 80% by mass of methanol (3 liters) at room temperature overnight. This process of extraction was carried out 3 times. The resulting extract was concentrated with the use of an evaporator until only a small amount of an aqueous solution (about 400 ml) was left behind. The remaining aqueous solution was subjected to extraction with n-hexane (400 ml×3) and chloroform (400 ml) to remove a lipid and chlorophyll. The aqueous phase of the extract was concentrated to about 50 ml. The concentrate of the aqueous phase was fractionated via column chromatography (column diameter: 7.3 cm; column length: 15 cm; support: Cosmosil 75C₁₈-OPN (Nacalai Tesque Inc.); mobile phase: methanol-water (0:100→400:0 (v/v))). As a result of preparative column chromatography described above, 13 fractions were obtained (Fractions 1 and 2: 0 vol. % methanol; Fractions 3 and 4: 5 vol. % methanol; Fractions 5 and 6: 10 vol. % methanol; Fractions 7 and 8: 20 vol. % methanol; Fractions 9 and 10: 40 vol. % methanol; Fractions 11 and 12: 60 vol. % methanol; Fraction 13: 100 vol. % methanol, with each fraction corresponding to a 500-ml eluate). These fractions were subjected to LC-MS analysis (Shimadzu LCMS-2020 system). As a result of LC-MS analysis, the molecular ion peak of m/z 307 [M+H]⁺ was detected primarily in Fraction 6 (10 vol. % methanol). Fraction 6 was separated with the use of the MS detecting fractionation system (Shimadzu LC-PDA-MS system). Fractionation conditions are as described below: LC-10AP Pumps A and B; column: Unison UK-C18 (150×10 mm (i.d.); 3 μm); column oven temperature: 40° C.; mobile phase: linear gradient elution, flow rate: 3 ml/min (Solvent A: water (0.1 vol. % HCOOH); Solvent B: acetonitrile (0.1 vol. % HCOOH); linear gradient program: 5 vol. % B (0 to 2 min), 5 to 8 vol. % B (2 to 3 min), 8 to 10 vol. % B (3 to 21 min), 10 to 100 vol. % B (21 to 22 min), 100 vol. % B (22 to 27 min), 100 to 5 vol. % B (27 to 28 min), 5 vol. % B (28 to 35 min)). In order to split the LC eluate to the LCMS-2020 mass analyzer at a split ratio of 1:150, a Makeup flow splitter (LC-10AD Pump C, Solvent B, 0.2 ml/min) was used. Electrospray ionization (ESI) mass spectra were recorded in the positive ion mode in the range of 100 to 1000 m/z (probe potential: 4.5 kV; nebulizer gas flow rate: 1.5 l/min; DL temperature: 250° C.; heat block temperature: 200° C.; dry gas flow rate: 20.0 l/min). As a result of preparative LC-MS described above, a compound exhibiting a molecular ion peak at m/z 307 [M+H]⁺ was obtained from Fraction 6 (an aqueous solution of 90 to 100 vol. % acetonitrile (0.1 vol. % HCOOH)) (61 mg). FIG. 1A schematically shows the purification process, and FIG. 1B shows the MS chromatogram obtained via fractionation using the preparative LC-PDA-MS system.

### [II: Determination of a Structure of a Novel Compound]

### [II-1: Determination of a Planar Structure of a Novel Compound]

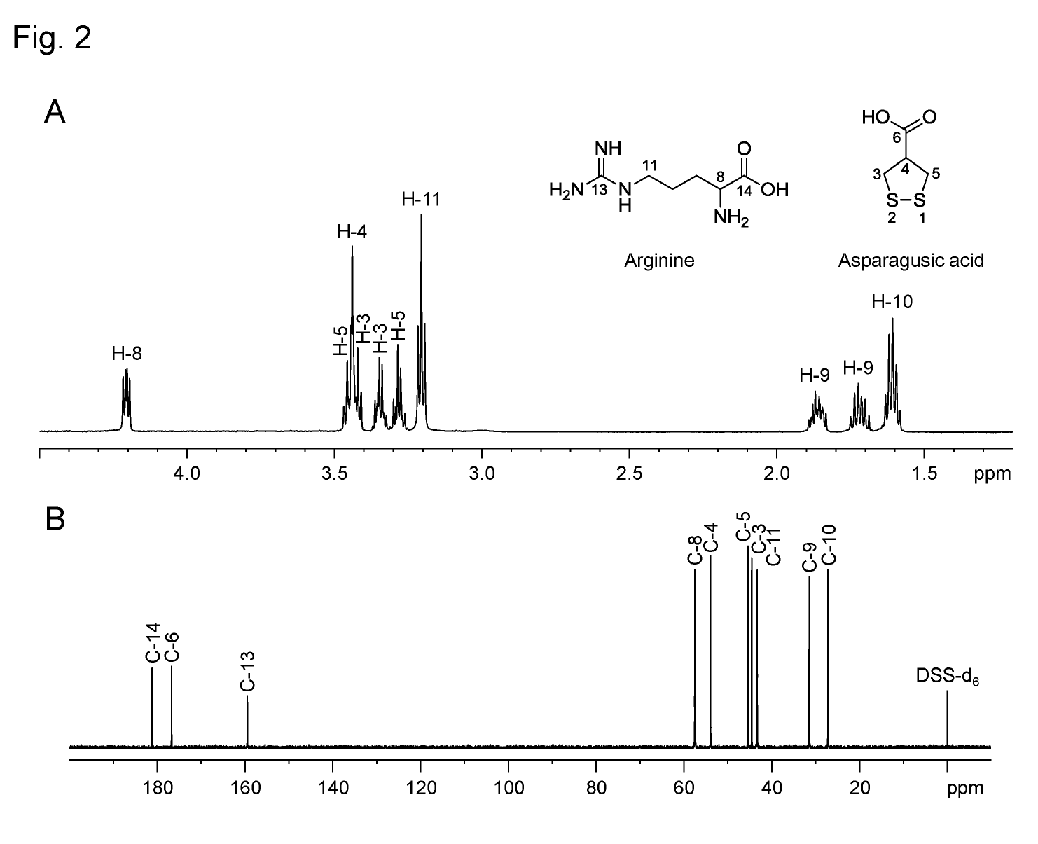

The spectral data of the compound isolated via purification were obtained. FT-ICR-MS: m/z 307.08931 ([M+H]⁺, calculated for C₁₀H₁₉N₄O₃S₂: 307.08930); ¹H-NMR (600 MHz, in D₂O containing 0.01% DDS-d₆, 25° C.), δ3.42 (1H, m, H-3a), 3.34 (1H, m, H-3b), 3.44 (1H, m, H-4), 3.46 (1H, m, H-5a) 3.28 (1H, m, H-5b), 4.21 (1H, dd 8.2, 5.0, H-8), 1.86 (1H, m, H-9a), 1.72 (1H, m, H-9b), 1.61 (2H, m, H-10), 3.21 (2H, t 6.9, H-11); ¹³C-NMR (150 MHz, in D₂O containing 0.01% DDS-d₆, 25° C.), δ44.6, 54.0, 45.4, and 176.7 (asparagusic acid portion: C-3 to C-6); 57.6, 31.5, 27.2, 43.3, 159.5, and 181.2 (arginine portion: C-8 to C-14). FIG. 2 shows the one-dimensional NMR spectra of the isolated compound. FIG. 2A shows the ¹H-NMR spectra and FIG. 2B shows the ¹³C-NMR spectra. In the heteronuclear multiple bond correlation (HMBC) spectra, the long-range correlation was observed between the proton signal (δ4.21 (H-8)) and the carbon signal (δ76.7 (C-6)).

On the basis of the spectral data, the planar structure of the isolated compound was determined as represented by Formula (I-1).

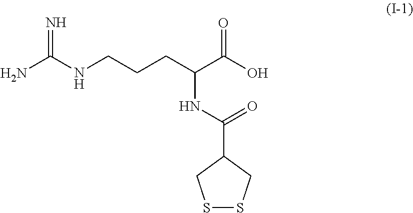 [SMILES: N=C(N)NCCCC(NC(=O)C1CSSC1)C(=O)O]

As shown in Formula (I-1), the isolated compound is a novel compound having a structure resulting from an amide bond formed between an α-amino group of arginine and a carboxyl group of asparagusic acid (Dawid, C. and Hofmann, T., J. Agric. Food Chem., 2012, Vol. 60, pp. 11877-11888), which are known compounds. The isolated novel compound was designated as asparaptine.

### [II-2: Determination of a Steric Structure of a Novel Compound]

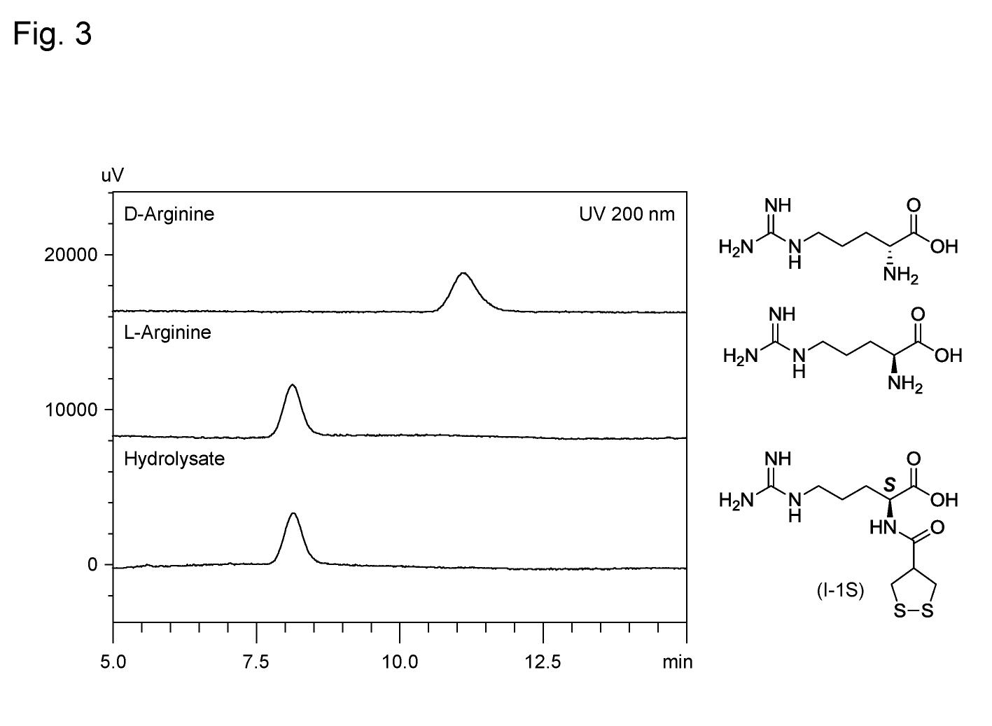

The absolute configuration of asparaptine at the alpha carbon position of arginine was determined in the manner described below. Asparaptine (1.9 mg) was suspended in 2.0 ml of 6.0 N HCl, and the suspension was heated at 85° C. for 8 hours. The resulting acid hydrolysate was dried under the nitrogen gas stream and then dissolved in water:methanol:HCOOH (30:70:0.02). The resulting solution sample was analyzed via HPLC under the conditions described below: LC-20AD Pumps A and B; column: Astec CHIROBIOTIC T (150×2.1 mm (i.d.), 1024AST); column oven temperature: 25° C.; solvent: (water:methanol:HCOOH (30:70:0.02)); flow rate: 0.2 ml/min; UV detection wavelength: 200 nm. The results of HPLC analysis are shown in FIG. 3. D-arginine and L-arginine indicated the retention time of 11.1 minutes and 8.1 minutes, respectively. L-arginine in the hydrolysate was identified in comparison with the retention time of an artificial sample. On the basis of the results described above, the absolute configuration of asparaptine at the alpha carbon position of arginine was determined to be an S arrangement as shown in Formula (I-1 S).

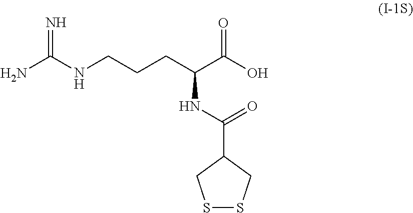 [SMILES: N=C(N)NCCC[C@H](NC(=O)C1CSSC1)C(=O)O]

### [III: Test of ACE Inhibitory Activity of a Novel Compound]

With the use of a commercially available kit for ACE inhibitory activity assay (ACE Kit-WST, A502, Dojindo Laboratories), ACE inhibitory activity of asparaptine, which is a novel compound represented by Formula (I-1S), was assayed. In principle, such kit enzymatically detects 3-hydroxybutyric acid (3HB) cleaved from 3-hydroxybutyryl-glycyl-glycyl-glycine (3HB-GGG) by ACE activity. In accordance with the instructions of the kit, a sample solution containing a test compound at a given concentration and a control solution with the addition of the same volume of water instead of the sample solution were prepared in each well of a 96-well microplate. An enzyme reaction solution was added to each well, and the reaction was then allowed to proceed. As a blank sample, a solution prepared with the addition of the same volume of water instead of the enzyme solution was subjected to the same treatment. The absorbance (450 nm) of the chromogenic indicator used in the enzymatic method generated together with 3HB cleaved from 3HB-GGG by ACE activity was measured. ACE inhibitory activity of each sample was determined using the formula shown below. A dose-response curve for ACE activity of each sample was prepared to determine the half maximal inhibitory concentration (IC₅₀ value) of each sample.

ACE inhibitory activity (%)={(blank absorbance−sample absorbance)/(control absorbance−blank absorbance)}×100

As samples to be assayed, L-arginine, D-arginine, L-aspartic acid, and asparagusic acid were used, in addition to the compound represented by Formula (I-1S) that was isolated in the manner described above (i.e., asparaptine). As positive control samples, captopril, N-succinyl-L-proline, and nicotianamine, which are known compounds having ACE inhibitory activity, were used. The structures of the compounds are shown below.

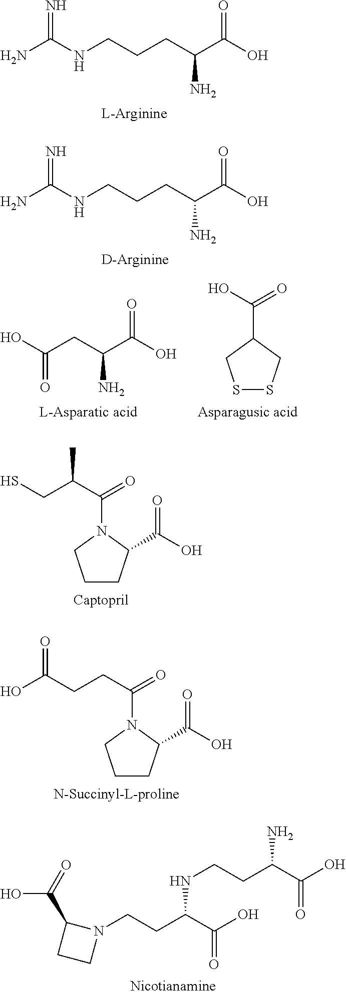

Table 1 shows IC₅₀ values concerning ACE activities of the samples. In the table, “N.D.” indicates that ACE inhibitory activity was not detected in the test.

TABLE 1

| Sample | IC₅₀ (μM) |
|---|---|
| Asparaptine | 113 ± 11 |
| L-Arginine | N.D. |
| D-Arginine | N.D. |
| L-Aspartic acid | N.D. |
| Asparagusic acid | N.D. |
| Captopril | 0.00161 ± 0.00043 |
| N-Succinyl-L-proline | 14.5 ± 5.3 |
| Nicotianamine | 18.7 ± 0.54 |

As shown in Table 1, asparaptine represented by Formula (I-1S) exerts ACE inhibitory activity at the concentration exceeding 1 μM and it exhibits IC₅₀ of 113 μM.

### [IV: Distribution of Novel Compounds in Asparagus Plants of Different Varieties or Those Grown Under Different Growth Conditions]

With the use of green asparagus grown in the light, white asparagus grown in the dark, and a purple asparagus variety containing a purple anthocyanin pigment, the amounts of asparaptine production were compared. In the same manner as described in I. above, asparaptine was purified from each fresh asparagus spear, and asparaptine contained in each purified fraction was quantified via LC-MS analysis. Table 2 shows the amount of asparaptine contained in each asparagus spear.

TABLE 2

| | Asparaptine content |
|---|---|
| Sample | (mg/kg fresh weight) |
| Green asparagus | 251 ± 17 |
| White asparagus | 298 ± 22 |
| Purple asparagus | 185 ± 26 |

As shown in Table 2, all asparagus spears were found to contain asparaptine.

All publications, patents, and patent applications cited herein are incorporated herein by reference in their entirety.

1. A compound represented by Formula (I-1): 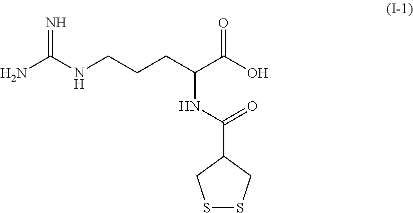 [SMILES: N=C(N)NCCCC(NC(=O)C1CSSC1)C(=O)O]
a salt thereof, or a solvate of the compound or the salt.

2.-4. (canceled)

5. A hypotensive food composition comprising the compound according to claim 1, a salt thereof, or a solvate of the compound or the salt, and one or more food industrially acceptable components.

6. A food composition comprising the compound according to claim 1, a salt thereof, or a solvate of the compound or the salt in an amount of 0.2% by mass or more, relative to the total mass, and one or more food industrially acceptable components.

7. A pharmaceutical composition comprising the compound according to claim 1, a salt thereof, or a solvate of the compound or the salt and one or more pharmaceutically acceptable components.

8. A method for producing the compound according to claim 1 comprising:
a step of extraction comprising subjecting asparagus to extraction with a water-miscible organic solvent; and
a step of isolation comprising separating the asparagus extract obtained with a water-miscible organic solvent in the step of extraction to isolate the compound according to claim 1 from the extract.

9. An asparagus extract obtained with a water-miscible organic solvent, which comprises the compound according to claim 1 in an amount of 0.01% to 90% by mass relative to the total mass of the extract obtained with a water-miscible organic solvent.

10. A method for producing the asparagus extract obtained with a water-miscible organic solvent according to claim 9 comprising:
a step of extraction comprising subjecting asparagus to extraction with a water-miscible organic solvent.

11. A method for prevention or treatment of the one or more diseases or symptoms comprising administering an effective amount of the compound according to claim 1, a pharmaceutically acceptable salt thereof, or a pharmaceutically acceptable solvate of the compound or the salt to a subject who is in need of prevention or treatment of the diseases or symptoms.

12. The method according to claim 11, wherein the diseases or symptoms are one or more diseases or symptoms selected from the group consisting of hypertension and cardiac failure.
